(19) United States
(12) Patent Application Publication
(10) Pub. No.: US 2023/0112155 A1
(43) Pub. Date: Apr. 13, 2023

(54) ANTENNAS WITH ION CLUSTER TYPE SENSORS

(71) Applicants: CENTRALESUPELEC, GIF-SUR-YVETTE (FR); CENTRE NATIONAL DE LA RECHERCHE SCIENTIFIQUE, PARIS Cedex 16 (FR); ECOLE NATIONALE SUPERIEURE DE CHIMIE DE RENNES, RENNES (FR); INSTITUT NATIONAL DES SCIENCES APPLIQUÉES DE RENNES (INSA), RENNES (FR); UNIVERSITE DE BRETAGNE OCCIDENTALE, BREST (FR); UNIVERSITÉ DE RENNES 1, RENNES Cedex (FR); UNIVERSITE DE NANTES, NANTES (FR)

(72) Inventors: Antoine Denis, RENNES (FR); David Rondeau, PACÉ (FR); Mohamed Himdi, RENNES (FR); Xavier Castel, DINAN (FR); Stéphane Cordier, PLELAN-LE-PETIT (FR)

(21) Appl. No.: 17/909,563

(22) PCT Filed: Mar. 1, 2021
(86) PCT No.: PCT/EP2021/055080

§ 371 Date: Sep. 6, 2022

(30) Foreign Application Priority Data
Mar. 6, 2020 (FR) FR2002272

(51) Int. Cl. G01N 27/02; G01N 33/00; B01L 3/00

(52) U.S. Cl. CPC G01N 27/023; G01N 33/0047; B01L 3/502715; B01L 2300/0636

(57) ABSTRACT

A sensor for detecting a volatile compound or gas. The sensor includes a transducer having: a planar resonator including a plurality of metal tracks, on a printed circuit, of the coplanar type; and a sensitive layer deposited on a predetermined portion of the resonator. The sensitive layer is configured so that the presence of a volatile compound or of a gas to be detected implies a modification of the permittivity of the sensitive layer resulting in a modification of the features of the sensor during its electrical power supply.

### FIELD

The invention relates to the field of detecting the presence of volatile compounds. The invention relates more particularly to the detection of volatile compounds by means of a wireless sensor.

### PRIOR ART

The detection of VOCs (Volatile Organic Compounds) began in the 1960s with the demonstration of the affinity of metal oxides with certain VOCs. The development of sensors based on the use of metal oxides was seen as essential in the monitoring of the environment, the control of chemical transformation methods, the safety of people, etc. Their study has contributed to the development of many manufacturing methods using metal oxide nanostructures of various geometries, such as nanoparticles, nanowires, nanorods and nanotubes whose dimension remains in the nanometric order. Due to the high surface/mass ratio, nanostructured metal oxides benefit from a strong interaction with the surrounding atmosphere and certain types of gases. However, their detection efficiency being strongly related to the manufacturing complexity of the sensitive layer, this makes these sensors expensive and limits their potential uses and deployments.

As illustrated in FIG. 1, a gas sensor of the prior art is composed of a selective sensitive layer and a transducer. The sensitive layer of the detector is at the heart of the device. It is this sensitive layer who is directly concerned by the phenomenon of sorption of the various gases. There are many natures of sensitive layers ensuring the detection of gas molecules. This detection can be more or less selective. The selectivity results in the ability of the sensitive layer to adsorb or absorb one or more volatile species. The existing sensitive layers are associated with a wide range of materials with specific properties. They are classified according to their chemical composition and divided into several categories such as metal/metal-oxide or polymeric nanostructures and organic-inorganic composites. As far as this last category is concerned, it is the technology of MOFs (Metal-Organic Frameworks), which is a real success today.

The sensitive layers presented previously allow the detection of many constituents present in the atmosphere, such as ammonia, hydrogen disulfite, nitrogen oxide, water, carbon monoxide, nitrogen dioxide, hydrochloric acid, as well as organic compounds (VOC) including solvents and pathogens, hydrocarbons, etc . . .

Conductive polymers have a strong affinity with VOCs, however the water naturally present in the environment destabilises the sensitive layer thus limiting the sensitivity of polymer technology sensors. Additionally, inorganic metal oxide gas sensors, such as tungsten oxide (WO₃), zinc oxide (ZnO), tin dioxide (SnO₂), titanium dioxide (TiO₂), iron oxides (Fe₂O₃/Fe₃O₄) and silicon dioxide (SiO₂), are related to a variable oxygen stoichiometry and to their surface electrical activity, increasing their detection capacity. Metal oxide gas sensors generally operate at high temperatures (200-400° C.), which can lead to drifts due to oxidation of the reference electrode. Despite high sensitivity, the implementations and applications of such sensors therefore remain limited despite everything. Their technological development is confronted with the properties of the sensitive organic layer which is both unstable and which has a highly limited electrical conductivity during operation at high temperature. Finally, the complexity of their implementation ultimately prevents low-cost industrialisation.

The use of nanocomposites combining two classes of materials allows, alternatively, to obtain gas sensors with improved and effective detection levels. Indeed, the design of nanocomposites seems to overcome the problems of stability due to the synergistic effects of the organic/inorganic base.

During the sorption of the gases on the sensitive layer of the sensor, certain physicochemical properties such as the complex impedance, the electrical conductivity, the thermal or optical features happen to be modified. The transducer has a central role in so far as it must convert a physico-chemical modification into a measurable quantity. The use of a transducer suitable for each type of physico-chemical variation is therefore very important in order to ensure suitable detection and requires an adapted design for the precise measurement of this variation. A wide variety of transducers exist. A brief presentation of said transducers is given in “Gas Sensors: A Review”, Sensors & Transducers, Vol. 168, Issue 4, April 2014, pp. 61-75.

The common point of these transducers is to lead to the detection of molecules in gas form. The problems associated with these sensors are diverse, ranging from significant manufacturing complexity to issues of cost and reliability and/or scaling up to industrial scale.

In addition, they require a physical connection to the electrical signal operating system. In order to transmit the information remotely, an antenna system must be added, further increasing the dimensions of the device.

### SUMMARY

The invention does not have these disadvantages of the prior art. More particularly, the invention relates to a sensor for the detection of volatile compounds or gases, comprising a transducer comprising:

a planar resonator comprising a plurality of metal tracks, on a printed circuit, of the coplanar type;

a sensitive layer deposited on a predetermined portion of said resonator;

said sensitive layer being configured so that the presence of a volatile compound or of a gas to be detected implies a modification of the permittivity of the sensitive layer resulting in a modification of the features of said sensor during its electrical power supply.

Thus, it is not necessary to have expensive equipment to carry out a detection of volatile compounds. Beyond the planar resonator, the described technique can be specified to microstrip type structures.

According to one particular feature, the power supply belongs to the group comprising:

a direct current (DC) power supply;

a radio frequency (RF) power supply; Thus, the power supply of the sensor is simple to implement.

According to a particular feature, the sensor takes the form of a communicating “senscillator”, comprising a two passively reconfigurable port antenna, integrated into a loop oscillator.

Thus, it is possible to precisely define operating modes of the sensor.

According to a particular feature, the loop oscillator comprises an amplifier using a field-effect or bipolar transistor, power supplied with direct current.

Thus, such a sensor can switch from an inactive state (in the event of absence of volatile compound to be detected) into an active state (causing the emission of an electromagnetic signal) which can be picked up.

According to a particular feature, the sensitive layer comprises molecular precursors of predetermined materials.

According to one particular feature, the molecular precursors based on metal clusters of materials comprise units with octahedral clusters with capped faces of the type:

[M₆Xⁱ₈L^{a}₆]^{n-/0/m+}

wherein:

M comprises W (tungsten) or Mo (molybdenum);

Xⁱ comprises Cl (Chlorine) or Br (Brome) or I (Iodine);

L^{a} comprises F (Fluor), X, functional acetates, functional phosphines, functional sulphates, NCS, H₂O, OH, or any other donor ligand.

According to one particular feature, the molecular precursors based on metal clusters of materials comprise units with octahedral clusters with bridged edges of the type:

[M₆Xⁱ₁₂L^{a}₆]^{n-/0/m+}

wherein:

M comprises Nb (Niobium) or Ta (Tantalum);

Xⁱ comprises Cl (Chlorine) or Br (Brome) or I (Iodine);

L^{a} comprises F, X, functional acetates, functional phosphines, functional sulphates, NCS, H₂O, OH, or any other donor ligand.

According to a particular feature, the molecular precursors based on metal clusters of materials comprise units of the type with octahedral clusters:

[Mo₆Xⁱ₈L^{a}₆]²⁻

wherein:

Xⁱ comprises Cl (Chlorine) or Br (Brome) or I (Iodine).

The invention also relates to a system characterised in that it comprises at least one sensor as previously defined.

According to a particular feature, such a system further comprises at least one device for measuring an electromagnetic signal emitted by said at least one sensor.

### DRAWINGS

Other features and advantages will appear more clearly upon reading the following description of a preferred embodiment, given by way of a simple illustrative and non-limiting example, and the appended drawings, among which:

FIG. 1, already presented, shows the operation of a gas sensor according to the prior art;

FIG. 2 shows the general principle of the invention;

FIG. 3 illustrates an embodiment of an antenna sensor;

FIG. 4 illustrates an embodiment of an improved quality factor antenna sensor;

FIG. 5 illustrates an embodiment of a passively reconfigurable closed-loop oscillator;

FIG. 6 illustrates an embodiment of a communicating “senscillator”, two passively reconfigurable port antenna integrated into a loop oscillator;

FIG. 7 illustrates an embodiment of a passively reconfigurable power divider.

FIG. 8 illustrates an exemplary embodiment of a system comprising a combination of sensors illustrated in the exemplary embodiments.

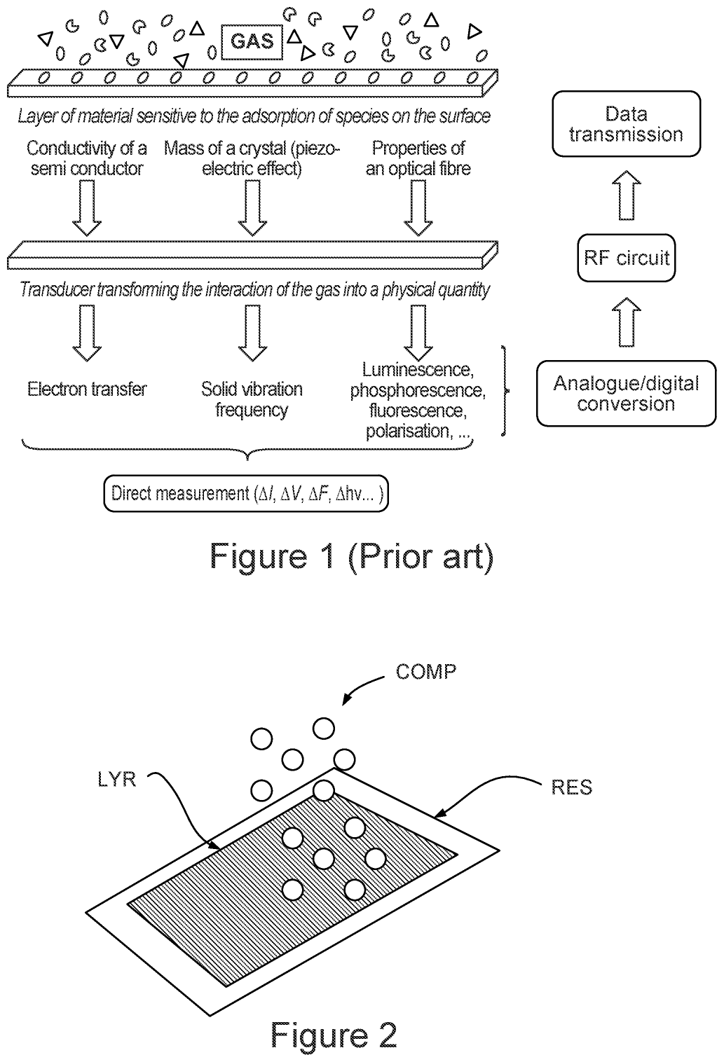

### DETAILED DESCRIPTION

Reminders of the Principle

As indicated previously, the sensors of the prior art do not offer a satisfactory solution, in particular in terms of cost and ease of use. An object of the invention is to provide a sensor of volatile compounds (gas, solvent) of small dimensions ensuring precise detection with high selectivity according to a wide range of gas molecules while being directly communicating with its environment, operating at room temperature, all at low cost. The proposed technique allows the detection and distinction of gas molecules by a device of reduced dimensions with increased sensitivity respecting selectivity and ensuring the direct transmission of measurements (wireless communicating sensors).

The general principle of the invention consists in providing a resonator (microwave), comprising predetermined features, with a layer of at least one specific material, thus delivering a transducer of the element to be detected towards microwave. This material is selected beforehand so that it interacts with a range of elements to be detected (for example a predetermined set of gases or volatile compounds). This interaction has the consequence of modifying the features of the transducer (and consequently the microwave features). These modified features are measured and reveal, if necessary, the presence of one or more types of elements (depending on the number of specific materials of the transducer). In other words, the invention generally relates to the production of a particular resonator to which a sensitive layer is added, which delivers a microwave transducer (also called an antenna sensor). FIG. 2 explains the general principle of the invention. The resonator (Res) is powered via a suitable power supply. On the resonator (Res), a layer (LYR) of specific material (based on a metal cluster for example) is deposited: the resonator has at this stage reference resonance features. When a volatile compound (Comp) is present, this compound alters the properties of the sensitive layer (LYR), which leads to a modification of the resonance features of the resonator (RES), and therefore a detection of the presence of the compound (Comp) by detecting the change of these resonance features: one passes from reference resonance features to different current resonance features. An indirect measurement of the presence of the volatile compound is therefore carried out. In other words, an object hereof is to use the surface and signal transduction properties from active surfaces modified by molecular ion deposition. For this purpose, clusters are used as a sensitive layer.

In a particular embodiment described below, this antenna sensor is based on a sensitive layer based on molybdenum clusters. Other clusters, such as tungsten or niobium or else tantalum clusters, can be used, depending on the ranges of volatile compounds to be detected. The layer is deposited for example by a technique of pneumatically assisted electro-nebulisation (electrospray). Clusters can be hexahedral or octahedral, depending on the chosen formulation. A sensitive layer can comprise a plurality of different types of clusters, in order to allow detection of several ranges of different elements.

The antenna sensor object of the present invention differs from the solutions of the prior art:
by the plural interactions of the molecules to be detected with the active surface (sensitive layer);
by the nature of the signal transduction directly associated with the activity of the active layer; and
by the wireless transmission of chemical information (indirect detection).

The operation of these sensors is indeed based on the polarisability of the different cluster units (for example molybdenum cluster, tungsten cluster, etc.) deposited on the sensitive surface. The sorption of the captured molecules (solvents, gas, . . . ) causes the modification of the polarisation of the cluster units, inducing a modification of the permittivity of the active layer, and consequently a modification of the microwave features of the transducer. Depending on the embodiments, the permittivity of the active layer evolves according to the presence or absence of compounds to be detected. For example, the inventors have determined that a dielectric permittivity close to 4.5 characterises a saturation of the sensitive layer by the element(s) to be detected. On the contrary, the antenna sensor of the invention is configured so that during its manufacture, the dielectric permittivity is around 2.5. This dielectric permittivity value is obtained by depositing a sensitive layer based on cluster materials of a predetermined thickness (for example 18 μm thick).

The antenna sensor comprises, in its simplest form, a transducer, of predetermined shape, depending on the needs; and a sensitive layer to be deposited on a specifically defined portion of the transducer. In operation, the sensor is power supplied (by an RF generator or a DC source or any other suitable device). The power supply causes the radiation of the transducer at least at a predetermined frequency (for example around 30 GHz) with specific features (for example reflection S₁₁, transmission S₂₁ coefficients as well as a predetermined gain). According to the invention, when the sensitive layer is altered by the sorption action of a compound to be detected on the sensitive layer, the features (frequency, reflection S₁₁, transmission S₂₁ coefficients and gain) are modified. These features can be measured to determine the presence or absence of one or more volatile compounds or predetermined gases (advanced system) or else the radiation can be used in a more basic way to generate a visual alert (simplified system). The device for measuring these features may or may not be integrated into the sensor itself, depending on the implementations. However, to favour production at a lower cost of the sensors, it is possible to have a measuring device independent of the sensor itself.

The interactions between captured molecules and the cluster units are of different natures: hydrogen bonding, electrostatic interaction and potentially chemical reactivity.

In addition, the chemical modification part of the sensitive layer being almost non-existent with regard to the effects of sorption, these sensors are reusable. Over a long term use, the sensor can be reconditioned: to reach saturation, the replacement of the sensitive layer is simple, fast and inexpensive, thanks to a technique of the “electrospray” type in order to redeposit a layer of nanosensors thus allowing to regenerate the antenna sensor.

If the electromagnetic wave EM/matter interaction is considered in the context of this antenna sensor, the determination of the nature of the molecule attached to the surface of the active layer is achieved for example through the simultaneous measurement of different complementary parameters: reflection coefficient S₁₁, transmission coefficient S₂₁ and gain. The tests carried out, in particular the indicative embodiments set out below, allow to conclude that there is good detectability in particular of the compounds: acetonitrile, ethanol, methanol, propanol, in particular by measuring the reflection coefficient S₁₁ which allows to distinguish the various compounds.

Moreover, the originality of the engineering of the antenna sensors object of the present disclosure consists in the fact that these chemical sensors are designed from molecular precursors based on metal clusters of materials with specific properties (for example molybdenum halide clusters) whose reactivity vis-à-vis gas molecules at atmospheric pressure, was previously revealed using chemical analyses by mass spectrometry, before shaping the sensitive surface.

More particularly, for clusters with capped faces [M₆Xⁱ₈L^{a}₆]^{n-/0/m+} wherein:
M comprises W (tungsten) or Mo (molybdenum);
X comprises Cl (Chlorine) or Br (Brome) or I (Iodine);
L comprises F (Fluor), X, functional acetates, functional phosphines, functional sulphates, NCS, H₂O, OH, or any other donor ligand,

The polarisation increases in the order:

M₆Iⁱ₈I^{a}₆<M₆Brⁱ₈Br^{a}₆<M₆Clⁱ₈Cl^{a}₆<M₆Brⁱ₈ (NCS)^{a}₆<M₆Br₈Cl^{a}₆<M₆I₈Br₆<M₆Iⁱ₈Cl^{a}₆<M₆Iⁱ₈(OOCR)^{a}₆<Mo₆Iⁱ₈F^{a}₆ where R=CₙF₂ₙ₊₁ and other electron-withdrawing groups.

The more polarised clusters react with the more polar gas molecules which are targeted to account for the composition of an industrial atmosphere in terms of detecting environmental pollution. Thus, the first clusters of the series above will interact preferentially with the first target molecules of the table below. The same goes for the most polar clusters which will detect the most polar molecules in the table.

TABLE 1
Polarity of molecules able to interact with a sensitive layer
Compounds characteristic of Industrial Toxic Compounds
and their dipole moment in Debye

Carbon tetrachloride (0) - Least polar molecule in the series
n-butylbenzene (~0.60)
Xylene (0.62)
Ethylamine (~1)
Chloroform (1.01)
Diethyl ether (~1.15)
Phenol (1.45)
Methylen Chloride (1.60)
n-Butyl acetate (~1.8)
Chloroacetophenone (~1.9)
Formaldehyde (2.33)
Dimethylformamide (3.86)
Dimethylsulfoxide (3.96) - Most polar molecule in the series

For clusters with bridged edges:[M₆Xⁱ₁₂L^{a}₆]^{n-/0/m+} wherein:
M comprises Nb (Niobium) or Ta (Tantalum);
X comprises Cl (Chlorine) or Br (Brome) or I (Iodine);
L comprises F, X, acetate, NCS, H₂O, OH, or any other donor ligand.

The polarisation increases according to the order: M₆Brⁱ₁₂Br^{a}6<M₆Clⁱ Cl^{a}₆<M₆Clⁱ (NCS)^{a}₆<M₆Brⁱ₈ (H₂O)^{a}₆<M₆Brⁱ₈ (OOCR)^{a}₆. Thus, the same order of cluster-gas molecule interaction occurs with this type of cluster.

Thus, it is proposed in an exemplary embodiment, to convert into electromagnetic radiation the chemical reactivity of anionic units of the types [Mo₆Xⁱ₈X^{a}₆]²⁻ (X═Cl, Br, I) with molybdenum (Mo) clusters such as nanosensors. The latter ensure the selectivity of the detection which results in a specificity of the information transmitted through complementary features (parameters) such as the reflection S₁₁ and transmission S₂₁ coefficients as well as the gain.

The direct association of the nanosensors in the form of an active layer with the microwave transducer ensures, on the one hand, a better sensitivity and selectivity to the nature of the volatile compound or the polluting gas and, on the other hand, greater reactivity associated with a direct transmission of information without the need to go through a related antenna system, or else through a wired connection (thus allowing to facilitate industrialisation and lower the cost of the sensor) and nor through an additional electronic implementation on the sensor itself. This autonomous antenna sensor also operates at room temperature and in the millimetre range (approximately 30 GHz), thus giving it reduced dimensions (active surface of a few mm²) for optimal integration in a system using in particular the frequency bands linked to the deployment of the 5G network.

More generally, according to the invention, beyond the halogenated units [M₆Xⁱ₈X^{a}₆]²⁻, the inventors have determined that units of the type [M₆Xⁱ₈L^{a}₆]^{n-/0/m+} (M=W and Mo; X═Cl, Br, I; L=F, X, functional acetates, functional phosphines, functional sulphates, NCS, H₂O, OH, or any other donor ligand) can be used as a nanosensor based on chemical reactivity to detect the target compounds.

The nature of the ligands X and L allows to modulate the polarisation properties of the cluster by the solvent molecules and therefore the detection performance of the associated microwave devices. It is thus possible to broaden the range of precursors of materials with bridged-edge units of the type [M₆Xⁱ₁₂L^{a}₆]^{n-/0/m+} with M=Nb, Ta; X═Cl, Br, I; L=F, X, functional acetates, functional phosphines, functional sulphates, NCS, H₂O, OH, or any other donor ligand, in order to further broaden the range of gases or volatile compounds targeted.

One of the particular advantages of the invention is that the proposed antenna sensor does not require a data processing processor. Indeed, the modification of the specific features (reflection, transmission coefficient, gain) of the signal generated by the antenna sensor is sufficient to ensure recognition of the presence of at least one element to be detected. It is thus possible to have a large quantity of antenna sensors, at low cost, allowing simple detection of one or more potentially dangerous compounds. The transducer, when it is defined, is shaped to favour electromagnetic radiation: the objective is to create a characteristic distribution of the signal so that it can be recognised (identified) according to the detection or not of one or more several chemical or organic compounds or elements.

### DESCRIPTION OF EXEMPLARY EMBODIMENTS

In this exemplary embodiment, a family of transducers associated with a sensitive layer ensuring direct and wireless transmission of information is described. These transducers are coated with an inorganic layer based on molybdenum clusters, sensitive to a range of solvents (methanol, ethanol, propanol, acetonitrile, etc.). In this exemplary embodiment, a granular morphology of the sensitive layer is preferred, thus optimising the specific surface of the sensitive layer, and therefore the surface exchanges between the solvent vapours and the sensitive layer: the granular layer ensures that the compounds to be detected will be in contact with a larger developed surface of the sensitive layer, which increases the detection sensitivity, because increases the interactions between the sensitive layer and the components to be detected.

The reactivity of the sensor works as follows: first, the solvent present in the surrounding atmosphere is adsorbed on the sensitive layer. This first effect causes a first shift in the operating frequency of the device on which the active layer is deposited due to the modification of the relative permittivity undergone by the latter. For example, in the presence of methanol, a chemical reaction occurs by the substitution of the chlorinated ligands (Cl) by the species (OCH₃)⁻ due to the reactivity of the clusters [Mo₆Clⁱ₈Cl^{a}₆]²⁻.

Interactions with molecules such as methanol are more related to interactions with clusters deposited on the active surface.

The electromagnetic features of the device (S₁₁, S₂₁, gain, . . . ) being directly related to its direct environment (that is to say the physico-chemical features of the sensitive layer), its performance is therefore modified and is sensitive to the nature of the solvent in interaction with the active layer (sensor selectivity).

In the interest of developing low cost devices, models of transducers have been designed such as microwave dipole resonators, printed on standard dielectric substrate and are presented below as exemplary embodiments of the present technique, without these embodiments being considered as limiting.

### Microwave Dipole Resonators

### Configuration 1

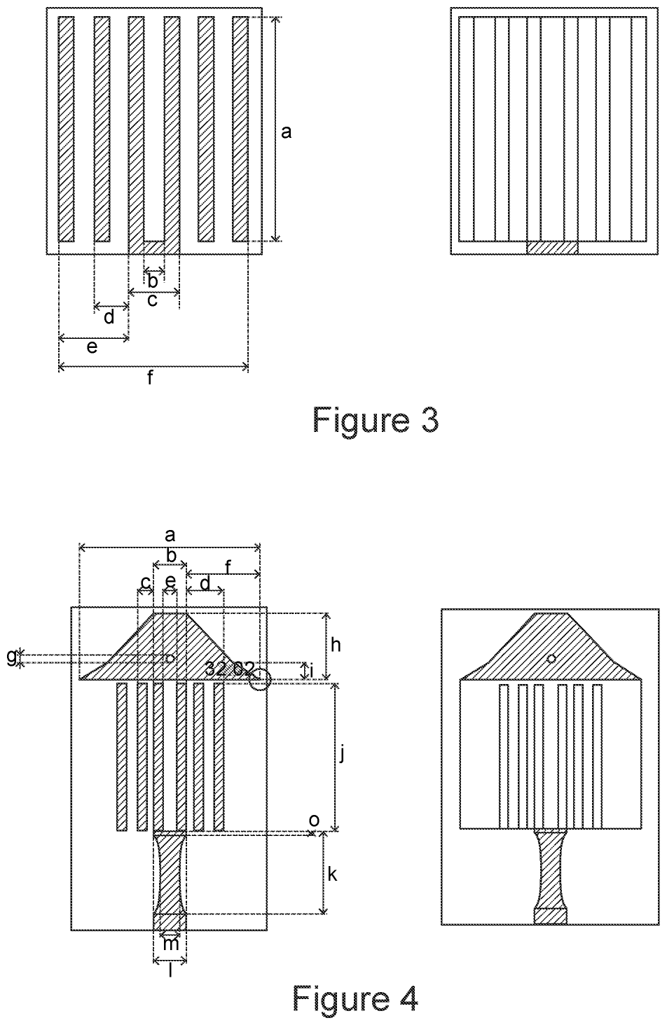

FIG. 3 shows a rectangular radiating element formed of six resonators. The transducer/antenna is power supplied by a 50 Ohm line. The radiating element is coated with a layer of sensitive material based on [Mo₆Clⁱ₈Cl^{a}₆]²⁻ clusters deposited by electrospray. The modification of the relative permittivity by the adsorption of a reactant pollutant causes a shift in the working frequency of f1 to f2. The radiation diagrams are also modified. This radiating element has in particular allowed to characterise, according to the invention, the permittivity of the active layer.

More particularly, the transducer in this exemplary embodiment is based on a combination of dipole resonators. The use of microstrip (or coplanar) technology allows to increase the coupling of the electromagnetic field with the active layer deposited on the surface of the transducer. In order to develop a low-cost device, the communicating transducer is printed on a 254 μm thick substrate (PTFE composite reinforced with glass micro-fibres) with an 18 μm thick copper metallisation. The 2.85 mm×3.37 mm rectangular radiating element (FIG. 3) is formed of six 220 μm wide copper strips, the central spacing is 330 μm followed right and left by a spacing of 167 μm, the last spacing measures 230 μm. The transducer/antenna is power supplied by a copper line with an impedance of 50 Ohm and 770 μm wide. The following table shows the dimensions of the transducer in this first embodiment.

TABLE 2
dimensions of the communicating
transducer (in mm) - Configuration no1

| a | b | c | d | e | f |
|---|---|---|---|---|---|
| 3.37 mm | 0.33 mm | 0.77 mm | 0.49 mm | 1.04 mm | 2.85 mm |

In this exemplary embodiment, the power supply of the sensor is for example carried out using an RF generator or another device allowing to obtain similar results. The power supply can be constant or carried out as needed (for example periodic detection phase).

### Configuration 2

FIG. 4 shows a new concept of communicating transducer allowing to improve the quality factor Q associated with the specific resonance of the device. The communicating transducer is a 4.19 mm×6.7 mm rectangular radiating resonant cavity. The upper reflector is digitally designed to show a strong impedance on the outside and a weak one in the centre with the idea of favouring the transverse propagation of the wave within the cavity. A quarter-wave adaptation of inverse elliptical geometry is placed at the input of the system. The indicative dimensions are shown in the following table. The communicating transducer is power supplied by a 770 μm wide 50 Ohm line.

TABLE 3
dimensions of the communicating transducer
(in mm)—Configuration n°2

| a | b | c | d | e | f | g | h | i | j | k | l | m | o |
|---|---|---|---|---|---|---|---|---|---|---|---|---|---|
| 4.19 | 0.77 | 0.39 | 0.84 | 0.33 | 1.71 | 0.2 | 1.53 | 0.42 | 3.37 | 1.8 | 0.77 | 0.45 | 0.1 |

The factor Q having been improved, it shows a strong potential difference at the electric and magnetic fields. This difference between high extreme values results in a resonance factor (factor Q) of very good quality. The factor Q of this communicating transducer generates a better sensitivity compared to the communicating transducer 1 by approximately 500%. The bare communicating transducer in this configuration n° 2 resonates at two frequencies in the 24-34 GHz band. A first resonance at 30.52 GHz with |S₁₁|=−21.1 dB and a second resonance present at 33.26 GHz with |S₁₁|=−8.3 dB. The communicating transducer covered with a dielectric layer of permittivity εᵣ=2.7 shifts the first resonance to 29.78 GHz and modifies the adaptation with |S₁₁|=−29.8 dB. The second resonance, in turn, goes to 31.74 GHz for an adaptation of −6.8 dB. With for example a permittivity value εᵣ=4.5 (representing the saturated active layer) the first frequency appears at 28.07 GHz with |S₁₁|=−13.3 dB and the second resonance at 30.41 GHz with |S₁₁|=−3.8 dB. These variation values allow to establish the sensitivity interval of the communicating sensor in this configuration n° 2 and it has, for example, a sensitivity associated with the variation in permittivity of the order of 6.35×10⁻⁹/Hz.

In this exemplary embodiment, the power supply of the sensor is for example carried out using an RF generator or another device allowing to obtain similar results. The power supply can be constant or performed as needed (for example periodic detection phase).

### Passively Reconfigurable Closed Loop Oscillator

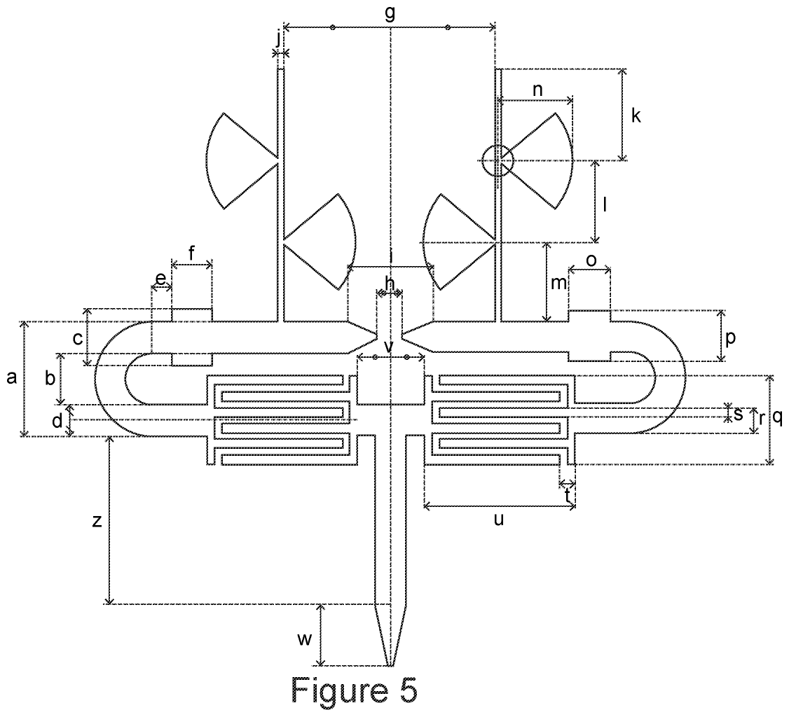

FIG. 5 shows a schematic description of a passively reconfigurable loop oscillator. This oscillator consists of an amplifier using a field effect or bipolar transistor power supplied with direct current (DC). Bare, it does not radiate and operates at a frequency close to 30.45 GHz. This oscillator, after deposition of the active layer on the interdigital capacitors, operates at a frequency close to 22 GHz. This oscillator allows the generation of a microwave signal when it is associated with an antenna. Subjected to one or more volatile compounds or predetermined gases, its working frequency increases. The sensitive layers are deposited in [A] and [B],

The dimensions are as follows:

TABLE 4
dimensions of the passively reconfigurable
closed-loop oscillator (in mm)

| a | b | c | d | e | f | g | h | i | j | k | l |
|---|---|---|---|---|---|---|---|---|---|---|---|
| 2.750 | 1.250 | 1.400 | 0.767 | 0.140 | 1.000 | 5.200 | 0.580 | 2.000 | 0.150 | 2.140 | 1.800 |
| m | n | o | p | q | r | s | t | u | v | x | z |
| 1.800 | 1.700 | 1.250 | 1.000 | 2.150 | 0.650 | 0.250 | 0.375 | 2.900 | 1.380 | 2.000 | 5.140 |

In this exemplary embodiment, the sensor is for example power supplied with direct current (battery, cell) or other devices allowing to obtain a direct current supply. The power supply can be constant or performed as needed (for example periodic detection phase).

### Communicating “Senscillator”

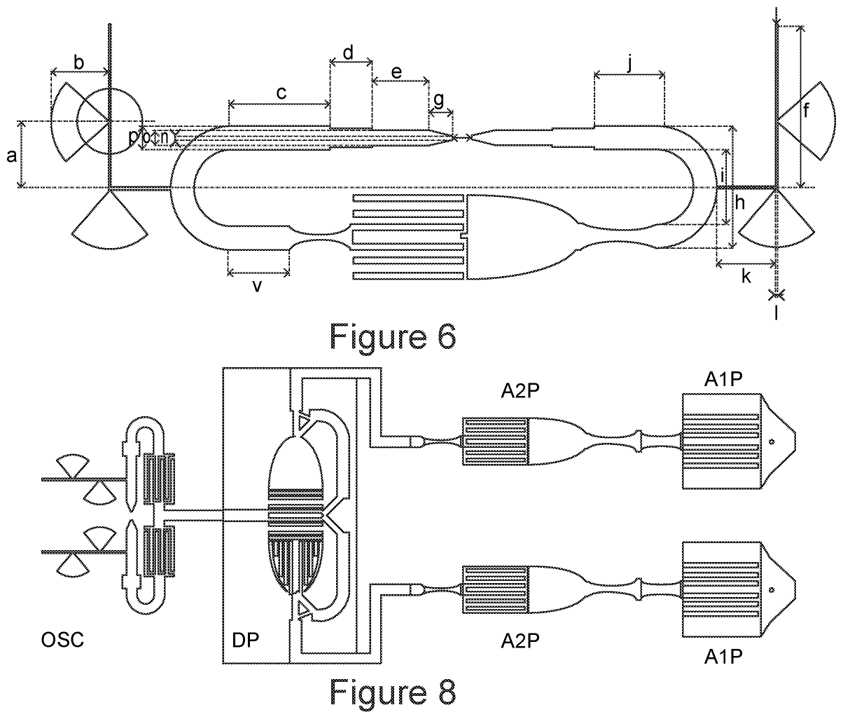

FIG. 6 shows a two (2) passively reconfigurable port antenna integrated into a loop oscillator, called a communicating “Senscillator”. Senscillator is a compound word from the Greek \ [symbol] \ describing here the “sense” of olfactory, touch type, etc. and \′αsIleIt\ describing the current swing mechanism, “oscillate” leading to the description of the word “oscillator” mechanism. Furthermore, Sens and Oscillator describe the term senscillator and these technological limitations. This oscillator consists of an amplifier using a field-effect or bipolar transistor power supplied with direct current (DC).

The radiating element [A] is coated with the active layer. During the modification of the relative permittivity due to the sorption of a gas by the active layer, a phase shift between the input and the output of the device causes the following operating scenarios:

The loop is designed so that after integration of the communicating sensor, it oscillates at 29 GHz. The job is to compensate for the phase effect added by the built-in communicating transducer. The parameters S after adding the 2-port communicating transducer evolve. It is observed that the |S₁₁| at 29.48 GHz is −5.2 dB, the |S₁₁| is at −10.1 dB and the |S₁₁| at −25.4 dB. The phase of the circuit displays 378° representing a slight shift in frequency. The gain is decreased from 4 dB previously to 0.2 dB.

TABLE 5
operating states of the communicating “senscillator”

| Initial state | Presence of detectable gas |
|---|---|
| Operation at Fl | Operation at F2 |
| Off | Operation at F1 |
| Operation at Fl | Off |

This allows to activate or deactivate the device in the event of a physico-chemical modification of the sensitive layer. A radiation diagram provides additional information regarding the state of the sensitive layer. This diagram can be used in the calibration phase for example, but it is not necessary to have it for a simplified version of implementation within a system.

In this exemplary embodiment, the sensor is for example power supplied with direct current (battery, cell) or other devices allowing to obtain a direct current power supply. The power supply can be constant or performed as needed (for example periodic detection phase).

### Passively Reconfigurable Power Divider

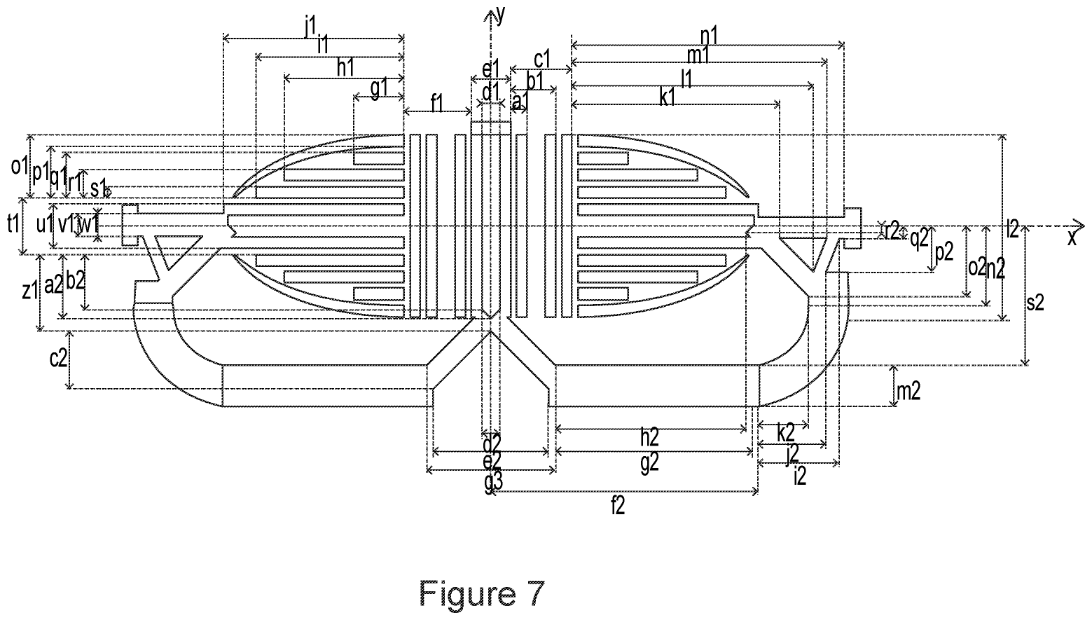

The circuit of FIG. 7 is a microwave power divider circuit. The power dividers called “conventional” power dividers have a division ratio R=P2/P3. The input adaptation must be ensured according to the ratio R. The modification of the division ratio generates a modification of the input adaptation of the device.

FIG. 7 presents a circuit with one input and two outputs, with three cavities, each having an overall surface equal to

$${( \frac{{λ}_{g}}{2} )}^{2}$$

The central cavity is made up of 100 Ohm microstrip line allowing to increase the coupling effects between the resonators at a frequency of about 30 GHz.
Two external cavities allow coupling with the outlet.

By depositing an active layer with a specific permittivity on one of the output cavities (for example on the output cavity on the left), the circuit becomes asymmetrical, and allows to control the power ratio R. It should be noted that the proposed power divider remains adapted regardless of the permittivity used in the interval comprised between 1 and 4.5 while modifying the division ratio R of the power divider.

TABLE 6
dimensions (in mm) of Figure 7.

| a1 | b1 | c1 | d1 | e1 | f1 | g1 | h1 | i1 |
|---|---|---|---|---|---|---|---|---|
| 0.311 | 0.861 | 1.172 | 0.33 | 0.77 | 1.27 | 0.96 | 2.28 | 2.83 |
| j1 | k1 | l1 | m1 | n1 | o1 | p1 | q1 | r1 |
| 3.45 | 4.39 | 4.58 | 4.84 | 5.16 | 1.19 | 0.97 | 0.86 | 0.54 |
| s1 | t1 | u1 | v1 | w1 | z1 | a2 | b2 | c2 |
| 0.22 | 0.435 | 0.87 | 0.42 | 0.42 | 1.48 | 1.19 | 1.03 | 1.07 |
| d2 | e2 | f2 | g2 | h2 | i2 | j2 | k2 | l2 |
| 0.296 | 2.14 | 5.1 | 3.79 | 3.64 | 1.54 | 1.28 | 0.95 | 3.44 |
| m2 | n2 | o2 | p2 | q2 | r2 | s2 | g3 | |
| 0.77 | 1.46 | 1.36 | 0.85 | 0.21 | 0.148 | 2.64 | 2.42 | |

In conclusion, this passively reconfigurable power divider allows the control of the power division while remaining adapted. The power division can be modified by the presence of the agent to be detected. The reconfigurable asymmetrical divider can be associated with an array of printed antennas in order to make its radiation unit agile (change of aperture at −3 dB for example). In this exemplary embodiment, the power supply of the sensor is for example performed using an RF generator or another device allowing to obtain similar results. The power supply can be constant or performed as needed (for example periodic detection phase).

### Implementation System

One or more sensors such as those previously presented can be implemented within a system comprising on the one hand an assembly comprising at least one sensor and an assembly comprising at least one network or spectrum analyser. The analyser is responsible for measuring predetermined parameters (equivalent to the parameters S previously described for the network analyser or else the radiation frequency for the spectrum analyser).

Nevertheless, a less expensive solution will favour the use of an RF oscillator associated with a detector of parameter S (in substitution for the network analyser), or a receiving antenna associated with a diode (in substitution for the spectrum).

More particularly, in at least one exemplary embodiment, a system which is the object of the invention comprises a communicating “senscillator” (for example that of FIG. 6, or any other communicating “senscillator” of the same type). Such a communicating “senscillator” is power supplied with direct current. When such a power supply is produced at the input and no gas is detected, the “senscillator” is in the OFF state (initial state) because the looping state is not achieved.

When there is presence of one or more volatile compounds or predetermined gases, the sensitive layer is altered and the scintillator operates (because the looping state is achieved due to the alteration of the sensitive layer) and it radiates at a frequency F1 (F1 depending for example on the volatile compound(s) detected). A receiving antenna (for example electrically unpowered in this exemplary embodiment) which converts the microwave signal F1 into a DC signal which power supplies one or more LEDs of predetermined colour: the colour can advantageously be representative of the volatile compound(s) detected, the voltage (DC) produced or the amount of current produced can possibly for example depend on the frequency F1 and therefore on the volatile compound(s) identified. A visual alarm is thus simply triggered. The system of the invention is therefore very inexpensive and very efficient and does not require any particular computing resources.

Other receiving antennas and/or devices for receiving the signals emitted by the communicating sensors presented can also be implemented according to requirements, for example by analysing the electromagnetic fields emitted by the sensitive layer sensors of the invention.

Such a system, according to an exemplary embodiment of the invention, is described in relation to FIG. 8. A system comprising a combination of sensors illustrated in the exemplary embodiments is more particularly presented. More particularly, in a logic of assemblable bricks, the various embodiments of the sensors presented above are assembled so as to produce a specific sensor “system” (SSS), allowing to meet particular needs in terms of generation of antenna diagrams for example. Thus, in the example of FIG. 8, the different functions implemented are:
A power divider (DP) with three (3) ports (corresponding to the example of divider in FIG. 7): in case of detection of a volatile organic compound, this sensor divides the distributed power asymmetrically;
Two (2) antennas (A1P) with one (1) port (as shown in FIG. 4): in case of detection of a volatile organic compound, the natural frequency of the antenna is modified, thus modulating the radiated power;
Two antennas (A2P) with two (2) ports (as shown in FIG. 4, also, with a series connection): these antennas act as a filter and an antenna, in case of detection of a volatile organic compound and modifies the natural frequency of the antenna and the transmission coefficient S₂₁.
An oscillator (OSC) (example in FIG. 5): in case of detection of a volatile organic compound, modifies the natural frequency of the oscillator which can go as far as extinction.

Thus, thanks to this combination, an autonomous radiating sensor is obtained: in case of detection of a volatile organic compound, modifies the natural frequency of the oscillator which can go as far as extinction, modifies at the same time the radiated power as well as the radiation frequency. Thus, according to the present disclosure, provision is made of a set of combinable sensors adapted to 50 Ohms. Other types of transducer can then be used under the condition of respecting the operating frequency as well as the port impedance standardised at 50 Ohm.

The advantage of this type of combination lies in the increase in sensitivity by cascade effect. Indeed, the generator generates a wave at a natural frequency for when a volatile organic compound is adsorbed on the sensitive layer, the frequency presented to the power divider changes from f₀ to f. The harmonic f is then transmitted to the power divider which when a V.O.C is adsorbed on its active layer becomes asymmetrical and allows to operate a power supply bias between the two outputs. The power supply of the connected antenna arrays is then asymmetrical. The adsorption of volatile organic compound on the sensitive layers of the antennas will modify the power accepted at the frequency f thus reducing the radiated power. The combination of the sensors then allows to obtain a very high gain in sensitivity. In addition, according to the present disclosure, it is not mandatory that each element of the system senses the same type of compound. Indeed, each sensor of such a system can optionally be configured to detect a particular volatile organic compound, among a given range of compounds. In addition, such a combinatorial antenna sensor system (ASS) can be integrated within a more global detection system. Such a global detection system comprises: an antenna sensor system, at least one device for picking up the signal emitted by the antenna sensor system, the device for picking up the emitted signal delivering data representative of picked-up signals (for example images of diagrams of antennas), these representative data can be obtained at different times (more or less regular time intervals, or in specific measurement situations). These representative data are then transmitted to an automated interpretation device (for example based on automated learning of the “machine learning” type). This automated interpretation device has been pre-trained to recognise, based on the representative data transmitted thereto, the presence or absence of volatile organic compounds. Such calibration of the system is for example carried out in advance, in the laboratory or in an industrial environment, to identify the presence of one or more types of V.O.C. The data necessary for the recognition (that is to say model) are directly implemented in the automated interpretation device so that it is operational.

1. A sensor for detection of a volatile compound or gas, comprising a transducer comprising:
a planar resonator comprising a plurality of metal tracks on a printed circuit; and
a sensitive layer deposited on a predetermined portion of said resonator;
said sensitive layer being configured so that presence of a volatile compound or of a gas to be detected implies a modification of permittivity of the sensitive layer resulting in a modification of features of said sensor during electrical power supply to the sensor.

2. The sensor according to claim 1, comprising a power supply input, which belongs to the group consisting of:
a direct current (DC) power supply input; and
a radio frequency (RF) power supply input.

3. The sensor according to claim 1, wherein the sensor is a communicating sensor, comprising a two passively reconfigurable port antenna, integrated into a loop oscillator.

4. The sensor according to claim 3, wherein the loop oscillator comprises an amplifier using a field-effect or bipolar transistor, having a direct current power supply input.

5. The sensor according to claim 1, wherein the sensitive layer comprises molecular precursors of predetermined materials.

6. The sensor according to claim 5, wherein the molecular precursors of predetermined materials comprise units with capped faces of a type:
[M₆Xⁱ₈L^{a}₆]^{n-/0/m+}
wherein:
M comprises W (tungsten) or Mo (molybdenum);
X comprises Cl (Chlorine) or Br (Brome) or I (Iodine);
L comprises F (Fluor), X, functional acetates, functional phosphines, functional sulphates, NCS, H₂O, OH, or any other donor ligand.

7. The sensor according to claim 5, wherein the molecular precursors with predetermined materials comprise units with bridged edges of a type:
[M₆Xⁱ₁₂L^{a}₆]^{n-/0/m+}
wherein:
M comprises Nb (Niobium) or Ta (Tantalum);
X comprises Cl (Chlorine) or Br (Brome) or I (Iodine);
L comprises F, X, functional acetates, functional phosphines, functional sulphates, NCS, H₂O, OH, or any other donor ligand.

8. The sensor according to claim 5, wherein the molecular precursors of predetermined materials comprise units of a type:
[Mo₆Xⁱ₈X^{a}₆]²⁻
wherein:
X comprises Cl (Chlorine) or Br (Brome) or I (Iodine).

9. A system comprising at least one sensor according to claim 1.

10. The system according to claim 9, wherein the system further comprises at least one device for measuring an electromagnetic signal emitted by said at least one sensor.
